(19) United States
(12) Patent Application Publication
(10) Pub. No.: US 2004/0023851 A1
(43) Pub. Date: Feb. 5, 2004

(54) Method for the porduction of artificial oxygen carriers from covalently cross linking haemoglobin with improved functional properties of haemoglobin by cross- linking in the presence of chemically non- reacting effectors of the oxygen affinity of the haemoglobin

(76) Inventor: Wolfgang Barnikol, Mainz (DE)

Correspondence Address: KURT BRISCOE, NORRIS, MCLAUGHLIN & MARCUS, P.A., 220 EAST 42ND STREET, 30TH FLOOR, NEW YORK, NY, 10017, US

(21) Appl. No.: 10/312,427

(22) PCT Filed: Jun. 2, 2001
(86) PCT No.: PCT/EP01/06329

(30) Foreign Application Priority Data
Jun. 29, 2000 (DE) 10031742.1

(51) Int. Cl. A61K 38/42; A61K 35/14

(52) U.S. Cl. 514/6; 530/385

(57) ABSTRACT

The invention relates to a method for the preparation of artificial oxygen carriers from chemically cross-linked hemoglobins with improved functional properties, chemically unreactive effectors of the oxygen affinity being added to the hemoglobin, before the latter is cross-linked and being present during the cross-linking. Pursuant to the invention, this leads to a reversible protection of those conformative regions of the hemoglobin molecules, which control the interactions of the hemoglobins with oxygen. This nonchemical protection causes the oxygen binding properties (especially the affinity and cooperativity) to change in a modified manner during a cross-linking of the hemoglobin's.

The invention relates to a method for the preparation of artificial oxygen carriers from chemically cross-linked hemoglobins with improved functional properties, chemically unreactive effectors of the oxygen affinity of the hemoglobins being added before the cross-linking of the latter and being present during the cross-linking. Pursuant to the invention, this leads to the reversible protection of those conformative regions of the hemoglobin molecules, which control the interaction between the hemoglobins and oxygen. This non-chemical protection causes the oxygen-binding properties (especially the affinity and cooperativity) to be modified during a cross-linking of the hemoglobins.

One reason for changing and modifying native hemoglobins chemically is to develop and produce artificial oxygen carriers from such hemoglobins. Unchanged, native hemoglobins, especially when dissolved in a functionally, highly effective molecularly dispersed form in the blood plasma, are not suitable as artificial carriers, because they are broken down by the kidneys into structural sub-units, which are rapidly excreted, because they continue also to leave the capillaries and because undesirable interactions with plasma proteins occur.

On the other hand, oxygen carriers are being developed, because very many and widespread pathological changes are based on an oxygen deficiency in tissues, in the acute case, after a heavy loss of blood—for example, after an accident or during major surgical interventions—and, in the chronic case, if there are circulation disorders, the latter, for example, in the case of so-called occlusive arterial diseases and, furthermore, in the case of a myocardial infarction, in the case of cardiac irregularities, in the case of a stroke, in the case of renal infarction, etc. Since artificial oxygen carriers are very efficient oxygen transporters, the diseases named can be controlled very effectively with them.

For developing artificial oxygen carriers, which do not have the above disadvantages, attempts must be made to realize conceptual approaches. The most important are:

The microencapsulation of hemoglobin solutions in liposomes so-called hemosomes (Ogata, Y. (1994): “Characteristics of Neo Red Cells, Their Function and Safety: In Vivo Studies”, Artificial Cells, Blood Substitutes, and Immobilization Biotechnologies 22: 875-881).

Covalent, intramolecular linkages, that is, a stabilization of the quaternary structure of the hemoglobins, either by bifunctional cross-linking agents (Farmer, M. C., et al. (1995): “Preclinical Data and Clinical Trials with Diaspirin Cross-Linked Hemoglobin”, Tsuchida, E. (ed.): Artificial Red Cells, John Wiley 1995: 177-185; Bakker, J. C., et al. (1988): “Properties of Hemoglobin Interdimerically Cross-linked with NFPLP”, Biomaterials, Artificial Cells, and Immobilization Biotechnologies 16: 635-636) or by obtaining appropriately changed hemoglobins by genetic engineering (Looker D. et at. (1992): “A Human Recombinant Hemoglobin Designed For Use as a Blood Substitute”, Nature 356: 258-260).

Covalent linkage of macromolecules, such as polysaccharides, dextrans, hydroxyethyl starch, inulin or artificial water-soluble macromolecules such as polyethylene glycols to the hemoglobin (Xue H. Wong J. T. F. (1994): “Preparation of Conjugated Hemoglobins”,—Abelson J. N., Simon, M. I. (Ed) Methods of Enzymology, Volume 231 B, Academic Press 1994: 308-322; Tam S. C. et al. (1978): “Blood Replacement in Dogs by DextranHemoglobin”, Canadian Journal of Biochemistry 56: 981-984; Patent DE-A 30 26 398 (1981): Modified hemoglobin-containing blood substitute”; EP-A 0 206 448 (1986) patent (1986): “Hemoglobin Combined with a Poly(alkylene Oxide)”, U.S. Pat. No. 5,234,903 (1993): “Chemically Modified Hemoglobin as an Effective, Stable, Non-immunogenic Red Blood Cell Substitute”, U.S. Pat. No. 5,312,808 (1994): “Fractionation of Polyalkylene Oxide-Conjugated Hemoglobin Solutions”).

Intermolecular cross linking (Gould S. A., et al. (1998): “The Clinical Development of Human Polymerized Hemoglobin”,-Chang, T. M. S. (Publisher): Blood Substitutes: Principles, Methods, Products and Clinical Trials, Volume 2, Karger Landes Systems 1998: 12-28; Pearce L. B. Gawryl M. S. (1998): “Overview of Preclinical and Clinical Efficacy of Biopure's HVOCs”,—Chang T. M. S. (Publisher): Blood Substitutes: Principles, Methods, Products and Clinical trials, Volume 2, Karger Landes Systems 1998: 82-98: Bakker J. C., et al. (1992): “Preparation and Characterization of Cross-linked and Polymerized Hemoglobin Solutions”, Biomaterials, Artificial Cells and Immobilization Biotechnologies 20: 233-241).

The last-named artificial oxygen carriers, which are based on crosslinked hemoglobins, have a series of advantages over the others. Sufficiently large cross-linked hemoglobins (hemoglobin polymers) have such a low colloidal osmotic pressure, that, when combined with a plasma expander, they can be used not only as an oxygen-transporting blood volume substitute to replace missing blood, but also added to the blood as oxygen-transporting blood additive (Barnikol, W. K. R., et al. (1996): “Hyperpolymeric hemoglobins as artificial oxygen carriers. An innovative approach to medical development” (Therapiewoche 46 811-815)). The treatment of many chronic oxygen deficient conditions, as mentioned above, is an area, in which the use of such oxygen-transporting additives is indicated. The treatment with an additive is always possible, even without a prior blood loss; on the other hand, all oxygen transporting (blood) volume substitutes named are suitable exclusively for the treatment of acute oxygen deficiency conditions after blood losses. Moreover, hemoglobins with a high degree of cross-linking have the advantage of a particularly long intravasal residence time. Furthermore, after their administration, an increase in blood pressure need not be expected since, because of their size, they do not leave the blood vessels and therefore cannot act as constrictors of the musculature of the vessels.

The normal supply of oxygen to the tissue by the natural hemoglobin, which is located in the red blood cells, is based essentially on the special oxygen-binding characteristics of hemoglobin as an S-shaped curve. These binding characteristics can be characterized by two parameters, namely by the so-called half saturation pressure of the oxygen (P50) as a measure of the average oxygen affinity on the hemoglobin, and the HILL index (n50) as a measure of the homotropic interactions or so-called cooperativity of the oxygen binding sites.

Both parameters determine decisively how effectively oxygen is absorbed from the air by the blood in the lungs and how effectively oxygen can be delivered from the blood in the capillaries to the tissue. Normally, the hemoglobin in human blood, packed in the red blood cells, has a P50 value of 25 torr and the high n50 value of 2.6; the corresponding values of the freely dissolved hemoglobin of man and the pig under physiological conditions are 16 torr and also 2.6. It has furthermore been known for a long time that the normal oxygen-binding characteristics are brought about in mammals in their red blood cells by small effector molecules, which can bind associatively (that is, not covalently) to the hemoglobin. In man and pigs, these effector molecules are 2,3-bisphosphoglycerate (DPG). This effector is bound, but not covalently, in the so-called central cavity of the hemoglobin molecule, which is formed by the 4 globular subunits of this molecule, which are disposed in a pseudotetrahydal arrangement; this bond is particularly stably in the de-oxygenated state of the hemoglobin. Aside from the natural effectors, foreign and artificial effectors are also known, which also effect the binding properties of human hemoglobin greatly, such as inositol hexaphosphate, inositol hexasulfate and mellitic acid, (W. K. R. Barnikol, O. Burkhard (1983): “The Fine Structure as an Adjuvant for Studying Pharmacological effects on the binding of oxygen to hemoglobin. Hemoglobin as a buffer of the oxygen partial pressure”. Funktionelle Biologie und Medizin 2: 245-249) evidently and obviously, such effectors bind specifically to the oxygen-dependent regions of the hemoglobin molecule.

Independently of the development strategy selected, it is important to be able to adjust the average affinity of molecularly dispersed artificial oxygen carriers to a particular, desired value and, as far as possible, to maintain the natural cooperativity at its high value.

The preparation of artificial oxygen carriers on the basis of crosslinked hemoglobin initially requires the covalent cross linking of hemoglobin into large molecules. Furthermore, these can then be linked covalently with inert and biocompatible molecules, in order to avoid undesirable interactions with plasma proteins. The cross-linking, as well as the covalent chemical-linking preferably take place at the amino groups of the hemoglobin molecules. If the chemical reactions mentioned are carried out with hemoglobins, undesirable changes in the oxygen affinity usually occur, especially a loss in the cooperativity (a decrease in the N50 value), for example, when the human hemoglobin is cross linked with divinylsulfone (H. Potzschke, St. Guth, W. K. R. Barnikol: “Divinylsulfone-Cross linked Hyperpolymeric Human Hemoglobin as an Artificial Oxygen Carrier in Anesthetized Spontaneously Breathing Rats: Advances in Experimental Biology and Medicine Vol. 345. Plenum Press, New York 1994: 205-214), especially if a high degree of cross-linking is to be achieved as far as possible without monomeric hemoglobin.

It is an object of the present invention to develop a method, for which, the changes, which arise during a cross linking of hemoglobin molecules, with respect to the affinity for the oxygen as well as the extent of the homotropic cooperativity of the oxygen binding sites of the cross-linked hemoglobins are prevented or modified in a desired manner.

Surprisingly, it was observed that known effectors of oxygen binding by hemoglobins can also be used to protect these hemoglobins during cross linking reactions, especially with glutardialdehyde, reversibly and effectively against a change in the oxygen affinity by the cross linking agent, especially for decreasing the reduction in the cooperativity.

Preferably, inositol hexaphosphate, inositol hexasulfate, mellitic acid and especially 2,3-bisphosphoglycerate are added as protection effectors pursuant to the invention preferably in amounts of 1 to 20, especially of 1 to 10, particularly of 1 to 3 and most particularly of 2 moles per mole of hemoglobin, which has not been cross-linked.

As hemoglobin starting material for the inventive teaching, monomeric native or chemically modified hemoglobin from man, from pigs or from cattle is suitable, human and particularly pig hemoglobin being preferred. The chemical modification of the hemoglobin may consist, for example, of a covalent linkage of certain low molecular weight materials, such as effectors of the oxygen affinity, for example, pyridoxal-5′-phosphat or 2-nor-2-formylpyridoxal-5′-phosphate (as described in Kothe et al. (1985), Surgery, Gynecology & Obstetrics 161: 563-569 or van der Plas et al. (1987), Transfusion 27: 425-430 and (1988), Transfusion 28: 525-530, further references in: Rudolph, A. S. et al (publishers): “Red Blood Cell Substitutes: Basic Principles and Clinical Applications”, Marcel Dekker, New York et al., 1998; Tsuschida E (publisher): “Blood Substitutes: Present and Future Perspectives”, Elsevier Science, Amsterdam 1998; Chang, T. M. S. (author and editor): Blood Substitutes; Principles, Methods, Products and Clinical Trials, Volume 1 and Volume 2, Karger Landes, Basel et al. 1997 and 1998, see also EP 0 528 841, in which the pyridoxylation of hemoglobin is described). Preferably, these molecules are attached before the inventive cross-linking.

Alternatively or additionally, a modification with molecules may be carried out, which improve the compatibility of the resulting artificial oxygen carriers with plasma. These include, for example, polyethylene glycols (survey in: Harris J. M. (Editor): Poly (Ethylene Glycol) Chemistry: Biotechnical and Biomedical Applications, Plenum, New York, et al. 1992). The reaction with this is described in the following. Furthermore, the hemoglobin preferably is deoxygenated or carbonylated by known procedures. Native, especially monomeric, hemoglobin from man and particularly from pigs, is preferred.

Cross-linking of monomeric hemoglobins with various cross-linking agents is known and repeatedly described in the literature. The following are given as examples. U.S. Pat. Nos. 4,001,200 and 4,001,401 relate to cross-linked hemoglobin as well as their use as a blood substitute and a plasma expander. The molecular weights (molar masses) of these cross-linked hemoglobins are between 65,000 and 1,000,000 g/mole. They can be synthesized by means of a plurality of cross-linking agents named, such as divinylsulfone, epichlorohydrin, butadiene epoxide, hexamethylene diisocyanate, the dialdehydes, glyoxal and glutadialdehyde, as well as the diimido esters namely dimethyl suberimidate and dimethyl malonimidate and dimethyl adipimidate.

Patent DE 24 49 885 relates, among other things, to cross-linked hemoglobins, which can be synthesized by reacting uncross-linked hemoglobins with various dialdehydes, such as malondialdehyde, succindialdehyde, glutardialdehyde, adipindialdehyde and suberdialdehyde.

U.S. Pat. No. 4,857,636 describes the synthesis of different crosslinked hemoglobins by the reaction of hemoglobin with various dialdehydes and polyaldehydes, for example, simple aldehydes such as glutardialdehyde and glyoxal, but also with structurally more complex aldehydes, which result from the oxidative ring opening of cyclic semiacetal or semiketal structures of the sugar molecules in monosaccharides and oligosaccharides as well as their derivatives.

U.S. Pat. No. 5,439,882 relates to cross-linked hemoglobins, which are synthesized by reaction with the dialdehydes, o-adenosine and o-ATP, formed by the ring-opening oxidation of the ribose in adenosine and in adenosine triphosphate. These cross-linked hemoglobins have molecular weights of 65,000 to 390,000 g/mole.

The EP 0 201 618 relates to a method of synthesizing extremely high molecular weight, soluble hemoglobin polymers, the so-called hyperpolymers, with molecular weight of 65,000 to 15,000,000 from highly concentrated solutions of monomeric hemoglobins.

The methods described are incorporated above. They can be employed pursuant to the present method, the unreactive cross-linking agent being added pursuant to the invention in the amount given immediately before the crosslinking reaction.

In principle, the cross linking of the hemoglobin takes place using a suitable polyfunctional or difunctional cross-linking agent for proteins such as butane diepoxide, divinylsulfone, a diisocyanate, especially hexamethylene diisocyanate, cyclohexyl diisocyanate or 2.5-bisisocyanatobenzenesulfonic acid, a di-N-hydroxy succinimidyl ester, a diimido ester, or a dialdehyde, especially glyoxal or the analogously reacting glycol aldehyde or glutardialdehyde. Cross linking with glutardialdehyde, as described in Potzschke, H. and Bamikol, W. (1992), Biomaterials, Artificial Cells, and Immobilization Biotechnology 20: 287-291 or as described in the following examples, is particularly preferred.

The cross linking agent is used in a molar excess of 3-fold to 60-fold and preferably of 6-fold to 35-fold, based on the monomeric hemoglobin, depending on the cross-linking agent. For example, a 7-fold and 10-fold molar excess of glutardialdehyde is preferred. Chemically unstable bonds, especially the Schiff's bases, which are formed by the reaction of functional aldehyde groups with amino groups of the hemoglobins, are stabilized reductively by known methods under suitable known conditions by reaction with suitable reducing agents, such as sodium borohydride in an adequate molar excess based on monomeric hemoglobin, a 2-fold to 100-fold and especially a 5-fold to 20-fold excess being preferred.

The cross-linking and derivatization reactions are carried out under conditions, which depend on the requirements of the chemical reactions selected. Native and modified hemoglobins are polyelectrolytes. Therefore, for reactions with the cross-linking agent, they are in aqueous electrolytes, which contain, for example salt and/or sodium hydrogen carbonate with ion concentrations of up to 300 mmoles/L and preferably between 50 and 200 mmoles/L. The reaction temperature for the cross-linking of the hemoglobin is 40 to 65° C., preferably 3° to 30° C. and especially 4° to 10° C. The proton activity in the solution, expressed as the pH is between 5 and 11, preferably between 6 and 9 and especially between 6.5 and 8. It can be adjusted to the desired value by known procedures, for example with lactic acid or sodium hydroxide solution. The effector is added to the reaction solution, which contains the still uncrosslinked hemoglobin in a concentration of 10 to 420 and especially of 150 to 400 g/L.

The hemoglobin can be deoxygenated, for example, by passing nitrogen and other oxygen-free inert gasses over the solution. Subsequently the cross linking agent is added in a known manner in a suitable molar ratio based on the monomeric hemoglobin. The reaction times depend on the special reaction selected, the temperature, the pH, the ion concentration, etc. and range from a few minutes up to 3 days and preferably are less than 5 hours and especially less than 2 hours. The excess of cross-linking agents can then be removed, for example, by reaction with suitable reducing agents or by physical methods.

The hemoglobins, so prepared, can optionally be modified further chemically, as mentioned below, for example, by linking polyalkylene oxides to them. Various methods of covalently linking polyalkylene oxides to proteins, especially also to uncross-linked hemoglobin, are known and described in the literature (the state of the art is comprehensively described by J. M. Harris (editor): Poly (Ethylene Glycol) Chemistry; Biotechnical and Biomedical Applications, Plenum, New York et al. 1992). In very many of these methods, the polyalkylene oxides are linked over a molecular bond (“spacer”), which is created, for example, by a difunctional linking agent. Strictly speaking, a product linking a polyethylene oxide by a cross-linking reagent to the protein is formed in these cases.

The linking of polyalkylene oxide to proteins is known (for example: U.S. Pat. No. 4,179,337 (1979): “Non-immunogenic Polypeptides”), especially also to hemoglobins, particularly also to artificial oxygen carriers based on modified hemoglobins (U.S. Pat. No. 5,478,805 (1995): “Fractionation of Polyalkylene Oxide-Conjugated Hemoglobin Solution”, U.S. Pat. No. 5,386,014 (1995): “Chemically Modified Hemoglobin As An Effective, Stable, Non-Immunogenic Red Blood Cell Substitute,” EP-A 0 206 448 (1986): “Hemoglobin Combined with a Poly (Alkylene Oxide)” EP-A 0 067 029 (1982): “Oxygen Carrier”). The contents of these publications are therefore incorporated here. However, according to the known literature, the linking of polyalkylene oxides to artificial oxygen carriers based on modified hemoglobins was never carried out with a cross-linked hemoglobin and was always intended to achieve completely different objectives, for example, a lengthening of the intravasal residence time or also a reduction in the immunogenic potency of artificial oxygen carriers.

For the covalent linkage of the polyethylene oxides, preferably those derivatives of polyalkylene oxides were used, which contain a cross-linking agent, with a functional group, which is already linked covalently, and reacts chemically directly with amino, alcohol or sulfhydryl groups of the hemoglobins with formation of a covalent linkage of the polyalkylene oxides, such as polyalkylene oxides with reactive N-hydroxysuccinimidyl ester, epoxide (glycidyl ether), aldehyde, isocyanate vinylsulfone, iodoacetamide, imidazolyl formate and tresylate groups, etc. Many such monofunctional, activated polyethylene glycols are commercially obtainable, for example, those named and having a molecular weight between 500 and 5000 g/moles.

Preferably, derivatives of a polyalkylene oxide, especially those selected from polyethylene oxide, polypropylene oxide or copolymers hereof, are used. Especially preferred are linkage products of polyalkylene oxide, especially those named, with a molecule, masking a terminal hydroxy group, especially as ether, ester, esteramide with short-chain (C₁-C₅) aliphatic organic group. Alternatively, non-active polyalkylene oxides, initially activated chemically in any further suitable manner or, possibly after an additional, necessary derivativization, are cross linked with the hemoglobin by chemical linking agents, for example, by a chemical reaction with bromocyan, a carbodiimide such as 1-ethyl-3-(3-dimethylaminopropyl)carbodiimide or N,N′-dicyclohexyl-carbondiimide, cyanuric chloride (polyethylene glycols, activated with the latter, as well as 4,6-dichloro-striazine polyethylene glycols, are also commercially available), or other known linking reagents, such as 2,2′-dichlorobenzidine, p,p′-difluoro-m,m′-dinitrodiphenylsulfone, 2,4-dichloronitrobenzine, etc. (survey in Harris J. M. (Publisher): Poly (Ethylene Glycol) Chemistry; Biotechnical and Biomedical Applications, Plenum, New York, et al. 1992).

Suitable as polyalkylene oxides are, in particular, polyethylene oxides (polyethylene glycols), polypropylene oxides (polypropylene glycols), as well as copolymers (mixed polymers) of ethylene oxide and propylene oxide, especially as already mentioned, certain derivatives of these, such as compounds masking an OH group, for example, (mono-) ethers with a short-chain alcohol, preferably with 1 to 5 carbon atoms, such as monoethyl ethers, monomethyl ethers, monopropyl ethers, etc., (mono-)esters with short-chain carboxylic acids, preferably with 1 to 5 carbon atoms, such as monomethyl esters, monoethyl esters, monopropyl esters, etc. and dehydration products with an aliphatic amine with 1 to 5 carbon atoms, such as monomethylamine, monoethylamine, monopropylamine, etc. with the one given above. Especially preferred are polyethylene glycols, and the derivatives of polyethylene glycol, which have been mentioned.

The molecular weight of the polyalkylene oxides used preferably is between 200 and 5,000 g/mole and especially between 500 and 2,000 g/mole. They are used preferably in an amount of 1 to 40 and particularly of 4 to 15 moles per mole of hemoglobin.

The linking reaction for the inventive procedure is carried out as described above. Accordingly, the hemoglobin can be linked with polyalkylene oxide with the help of known methods, as described above, for example, by direct combination with the help of a condensation agent, such as bromocyan, or with the help of a cross-linking reagent, such as cyanuric chloride (see DE-OS 30 26 398), or by reaction with an activated polyalkylene oxide, such as an N-hydroxysuccinimide ester of a polyalkylene oxide derivative. In this way, at least 1, especially 1 to 40 and preferably from 4 to 15 molecules of the polyalkylene oxide, used pursuant to the invention, is linked per molecule of monomeric hemoglobin.

For example, the following methods can be used to link the polyalkylene oxides, their structural integrity being retained:

(1) (Not activated) polyethylene glycol is reacted with the 2-fold to 5-fold molar amount and preferably the 3-fold molar amount of bromocyan at a pH of 9 to 10. The remaining bromocyan is removed from the reaction mixture by gel filtration, dialysis, etc. and the product is then reacted with the required amount, such as the 0.1-fold to 0.002-fold and preferably the 0.02-fold to 0.01 fold molar amount of hemoglobin at a pH of 7 to 9 and preferably of 7.5 to 8, in an aqueous solution (see DE-OS 30 26 398).

(2) Polyethylene glycol is added in benzene, which contains an excess of sodium carbonate, and then is reacted with the 2-fold to 5-fold and preferably the 3-fold to 4-fold molar amount of cyanuric chloride. The reaction product, polyethylene glycol-4,6-dichloro-s-triazine is removed and reacted with the desired amount of, for example 1 to 0.002 moles and preferably 0.1 to 0.01 moles, based on a mole of the reaction product named above, of hemoglobin in a buffer solution with a pH of 8 to 9.5 (see DE-OS 30 26 398).

(3) Activated polyalkylene oxide, such as an N-hydroxysuccinimide ester of a polyalkylene oxide, is added in a 1-fold to 40-fold excess, based on monomeric hemoglobin to an aqueous solution with a pH between 7 and 10 of a hemoglobin, which is to be linked to the polyalkylene oxide, and allowed to react.

The methods, explained above, can also be used in the case of the other polymers, used pursuant to the invention.

The polyethylene oxides are linked chemically to the artificial oxygen carriers of cross-linked hemoglobin in the course of the preparation of the inventive hemoglobin derivatives at three times.

i) In the first case, the polyethylene oxide derivative is linked to the native or modified hemoglobins (hemoglobin monomers) of high purity; subsequently the hemoglobins are cross-linked, particularly with a difunctional, cross-linking agent.

ii) In the second case, polyethylene oxide derivatives are linked to the already synthesized cross-linked hemoglobin, that is, subsequent to the reaction of the highly pure, native hemoglobin monomers or to the hemoglobin monomers modified with effectors, with a difunctional cross-linking agent.

iii) In the third case, finally, polyalkylene oxide derivatives can be linked covalently to hemoglobin monomers before the latter are cross-linked, as well as, additionally, thereafter, in the further course of the preparation, to the cross-linked hemoglobin.

As mentioned, an effector is added, pursuant to the invention, in each case before the cross-linking. Preferably, the modification is carried out with a polyalkylene oxide, especially a polyethylene oxide or its derivatives after the inventive cross linking, as mentioned above.

The cross-linked hemoglobins obtained can then be purified in a suitable, known manner, for example by centrifugation, filtration or ultrafiltration or chromatographically (for example, by preparative, volume-exclusion chromatography on, for example, Sephadex G-25 gel) or as described in the publication named above or in Curling, J. M.: Methods of Plasma Protein Fractionation, Academic Press, London, 1980) or EP-A 0 854 151, EP-A 95 107 280 and subsequently processed further to a pharmaceutical preparation.

Preferably, monomeric hemoglobin, preferably in the deoxygenated state, in an aqueous electrolyte (which contains, for example, sodium hydrogen carbonate or sodium chloride or sodium lactate or several of these), is initially mixed in the given amounts with one of the effectors named and subsequently cross linked, for example, with said difunctional cross linking agent, especially with a 7-fold to 10-fold molar excess of glutardialdehyde. Excess glutardialdehyde is removed by known procedures with sodium borohydride, for example, by the addition of a 2-fold to a 100-fold and especially of a 5-fold to a 20-fold molar excess, based once again of the monomeric hemoglobin. The crosslinked hemoglobins, so obtained, can then subsequently be worked up for example by dialysis, centrifugation, clarifying filtration, ultrafiltration, precipitation, for example with polyethylene oxide, preparative chromatographic methods, such as gel permeation, chromatography, and also processed further to a pharmaceutical preparation as an artificial oxygen carrier or also reacted with polyethylene oxide and worked up and then processed into a pharmaceutical preparation.

In this way, a cross-linked hemoglobin is obtained as product which, due to the presence of the unreactive effector during the cross-linking, has particularly advantageous oxygen affinity properties because a loss of cooperativity is avoided or reduced. In view of the state of the art, this advantage, achieved pursuant to the inventive teachings, was surprising, since a treatment with unreactive effectors is not known in the art and an effect of such unreactive molecules during the chemical reactions especially with highly reactive cross linking agents could not have been anticipated. The hemoglobins, cross linked pursuant to the invention, have an extremely advantageous, unexpected advantage, namely, a decrease in the loss of cooperativity of the oxygen binding in comparison with that of hemoglobins, cross linked quite similarly, but without the inventive procedure.

If polyakylene oxides are linked covalently as well to the cross linked hemoglobins, a distinctly improved plasma compatibility can be achieved in addition, even under extreme physiological conditions especially of the pH. Moreover, the compatibility is independent of the nature and of the molecular weight of the hemoglobin and of the cross-linking agent, effectors or polyalkylene oxide used.

The hemoglobin derivatives, prepared pursuant to the invention, can be used as such or in a form of suitable, for example, pharmaceutical preparations as artificial oxygen carriers, intravasally as pharmaceutical products or for biomedicinal purposes, as a replacement for blood for the treatment of a blood volume deficiency, as an addition to the blood for the treatment of pathogenic oxygen deficiency conditions, or as a nutrient solution in the human or animal organism, in organs or in biotechnical applications. In order to prepare the products, which are to be administered, the inventive hemoglobin products are dissolved in suitable media, such as infusion solutions, for example, in aqueous salt solutions or glucose solutions, both preferably in a concentration isotonic with the blood plasma.

Specially preferred developments of the invention are described in greater detail in the following, initially by means of a general preparative method and subsequently by examples.

Pig, human or bovine hemoglobin, native or after a prior chemical modification, for example with a covalently binding effector of the oxygen affinity and/or a covalently binding polyethylene oxide to improve the compatibility with plasma proteins, with a concentration between 10 and 40 g/L and preferably between 150 and 400 g/L, is dissolved in an aqueous sodium hydrogen carbonate solution having a concentration of 40 to 100 mmoles/L and a temperature ranging from 3° to 30° C. By passing pure nitrogen over this stirred hemoglobin solution, the hemoglobin is deoxygenated. The pH of the solution is adjusted with lactic acid or sodium hydroxide solution, having a concentration between 0.1 and 1 mole/L, to a value between 6 and 9 and preferably between 6.5 and 8. The addition of 1 to 10 and preferably 1 to 3 moles of the effector per mole of monomeric hemoglobin, which is to be cross-linked, now takes place, 2,3-bisphosphoglycerate being a particularly preferred effector. Subsequently, the reaction of the hemoglobin with a difunctional cross linking agent, selected from butadiene diepoxide, divinylsulfone, a diisocyanate, especially hexamethylene, diisocyanate, cyclohexyl diisocyanate and 2,5-bisisocyanatobenzenesulfonic acid, a di-N-hydroxysuccinimidyl ester, a diimido ester, or a dialdehyde, especially glyoxal, the analogously reacting glycol aldehyde and, in particular, glutodialdehyde, is carried out. The molar ratio of cross-linking agent to monomer hemoglobin is between 3 and 60 and preferably between 6 and 35 moles per mole of the monomeric hemoglobin. After cross linking with one of the dialdehydes named, the resulting Schiff's bases are reduced with sodium borohydride in a molar ratio to the monomeric hemoglobin of between 2 and 100 and preferably between 5 and 20. This reduction takes place at a pH of between 7.5 and 10 and preferably between 8 and 9; as described above, the pH is adjusted to this value with sodium hydroxide solution or lactic acid.

After the pH is adjusted once more to a value between 7.5 and 10 (with sodium hydroxide solution or lactic acid), the cross-linked hemoglobins can now be linked covalently with a polyalkylene oxide derivative in that the latter is added to the reaction mixture in a molar ratio of 1 to 40 and preferably of 4 to 15 to the monomeric hemoglobin. For the reaction especially with the amino groups of the hemoglobins, the polyalkylene oxides can already be activated monofunctionally or linked actively or passively. Without the preferred linking of the polyalkylene oxide, the further working up takes place directly.

The preferred, special procedure, on which also the Examples 1 to 3 below are based, is explained in the following.

Pig hemoglobin or human hemoglobin, which is dissolved at a concentration of about 150 to 400 g/L in an aqueous hydrogen carbonate (NaHCO₃) electrolyte having a concentration preferably of 40 to 100 mmoles/L and a temperature of 3° to 30° C., is deoxygenated by passing nitrogen over the stirred solution. Subsequently, preferably between 2 and 6 moles of sodium ascorbate per mole of hemoglobin are added and the solution is titrated with lactic acid to a pH preferably between 6.5 and 8. To the solution with the pH so adjusted (see Examples 2 and 3), one of the above-named effectors of the oxygen affinity is added in the amount given, preferably in an amount of 1, 2 or 3 moles per mole of monomeric hemoglobin. Subsequently, the cross linking is carried out by the addition especially of glutardialdehyde in a molar ratio of preferably between 7 and 10 moles per mole of monomeric hemoglobin. After the solution is titrated once again with sodium hydroxide solution (NaOH) to a pH preferably between 8 and 9, the Schiff's bases formed are reduced with sodium borohydride, which is added in a molar ratio preferably of 5 to 20 moles per mole of monomeric hemoglobin. With a further titration with sodium hydroxide solution or lactic acid, the pH is brought to a value preferably between 8 and 9 and activated polyethylene glycol such as an N-hydroxysuccinimidyl derivative with a molecular weight of 300 to 5,000 and preferably of 500 to 2,000 g/mole, is added in a molar ratio of 1 to 15 moles per mole of monomeric hemoglobin. Subsequently the resulting cross-linked hemoglobin is liganded with oxygen by passing pure oxygen over the stirred solution. For characterizing the oxygen binding properties under physiological conditions, the solvent, together with all the unconsumed reactants and the reaction products contained therein, is exchanged, for example, with the help of volume exclusion chromatography or ultrafiltration for an aqueous electrolyte, which corresponds to the plasma fluid and contains 125 mM of sodium chloride, 4.5 mM of potassium chloride and 20 mM of sodium hydrogen carbonate.

The invention is explained in greater detail by means of the following Examples. Example 1, in which unreactive effectors of the oxygen affinity have not been added for the cross linking of the hemoglobin, is a comparison example and Examples 2 and 3 represent an invention to demonstrate the improvements achievable.

### EXAMPLE 1

### Comparison for 22° C.

### Preparation of a Cross-Linked Hemoglobin Without the Inventive Addition of Unreactive Effectors of the Oxygen Affinity During the Cross Linking of the Hemoglobin at 22° C.

A 35% solution of pig hemoglobin (Hb) and 50 mM of sodium bicarbonate was deoxygenated by passing nitrogen over the stirred solution. Subsequently, sodium ascorbate was added (4 moles/mole of hemoglobin). The pH of the solution was then titrated with lactic acid to a value of 7.2 and treated with glutardialdehyde (9 mole/mole of hemoglobin) for a period of about 1.5 hours. After titration with sodium hydroxide solution (NaOH) to a pH of 7.8, the Schiff's bases formed were reduced with 10 moles of sodium borohydride per mole of hemoglobin for a period of 0.75 hours. After a further titration of the solution with sodium hydroxide to a pH of 8.5, the dissolved hemoglobin was treated with an 8-fold molar excess of methoxy-succinimidyl propionate polyethylene glycol, having a molecular weight of 2,000 g/mole, for a period of 1 hour. The cross linked hemoglobin was then liganded with pure oxygen. The milieu of the dissolved product subsequently was exchanged with the help of volume exclusion chromatography (Sephadex G-25 gel, Pharmacia, Germany) for a aqueous electrolyte solution having a composition of 125 mM of sodium chloride, 4.5 mM of potassium chloride and 20 mM of sodium bicarbonate.

Measurements under physiological conditions (37° C.), 40 torr carbon dioxide partial pressure and a pH of 7.4) revealed an n50 value (cooperativity) of 1.35 at a p50 value (average affinity) of 27 torr for the product.

### EXAMPLE 2

### Inventive Preparation of a Cross-Linked Pig Hemoglobin with the Addition of Unreactive Effectors of the Oxygen Affinity Before the Cross Linking of the Hemoglobin

The product was prepared in the same way as in Example 1 with the single exception that, before the cross-linking with glutardialdehyde, 3 moles of 2,3-bisphosphoglycerate per mole of monomeric hemoglobin, which is to be cross linked, were added. Under the aforementioned physiological conditions of the characterization, there was a clear decrease in the loss of cooperativity of the oxygen binding sites of the hemoglobin with an n50 value (as a measure of the cooperativity) of 1.7 with an increased, average oxygen affinity, expressed as a p50, of 18 torr.

### EXAMPLE 3

### Comparison for 4° C.

### Inventive Preparation of a Cross-Linked Pig Hemoglobin Without the Inventive Addition of an Unreactive Effector at 4° C.

The product was prepared in the same was as in Example 1 with the difference that the preparation was carried out at 4° C. and the reaction time prolonged 10-fold.

Under the aforementioned conditions, a p50 value of 38 torr and an n50 value of 1.1 were obtained.

### EXAMPLE 4

### Inventive Preparation of a Cross-Linked Pig Hemoglobin with the Addition of an Unreactive Effector of the Oxygen Affinity Before the Cross Linking

A polymeric hemoglobin was prepared by a method, quite similar to that described in Example 3, with the change that 2 moles of 2,3-bisphosphoglycerate per mole of hemoglobin were added before the cross-linking of the hemoglobin.

The n50 value was 1.7 at a p50 value of 19 torr.

### EXAMPLE 5

### Preparation of a Cross-Linked Pig Hemoglobin with the Addition of an Inventive, Unreactive Effector Before the Cross Linking

The product was prepared in the same way as in Example 3, with the exception that 2 moles of inositol hexaphosphate per mole of hemoglobin were added before the cross-linking.

Under the above-mentioned conditions, an analysis showed a p50 value of 17.5 torr and an n50 value of 1.2.

### EXAMPLE 6

### Preparation of a Cross-Linked Pig Hemoglobin with the Addition of an Inventive, Unreactive Effector Before the Cross Linking

The product was prepared by the method of Example 3 with the change that, before the cross linking, 2 moles of mellitic acid were added per mole of hemoglobin.

The analysis under the conditions named above showed the p50 value to be 14.6 torr and the n50 value to be 1.5.

### EXAMPLE 7

### Comparison Example

### Preparation of a Cross-Linked Pig Hemoglobin Without the Inventive Addition of an Unreactive Effector

The product was prepared as in Example 1, with the difference that the cross-linking agent, glycol aldehyde, was used in a 20-fold excess, the reaction time of the cross-linking was 4 hours and the pH being 9.1.

Under the conditions given, the analysis revealed a p50 value of 18 mm of Hg and an n50 value of 1.2.

### EXAMPLE 8

### Preparation of a Cross-Linked Pig Hemoglobin with the Addition of an Inventive, Unreactive Effector

The product was prepared as in Example 7 with the exception that 2 moles of 2,3-bisphosphoglycerate per mole of hemoglobin was added to the reaction mixture.

The analysis under the conditions given revealed a p50 value of 13 mm of Hg and an n50 value of 1.55.

### EXAMPLE 9

### Preparation of a Cross-Linked Pig Hemoglobin with the Addition of an Inventive, Unreactive Effector Before the Cross Linking

The product was prepared as in Example 7, with the difference that 2 moles of inositol hexaphosphate per mole of hemoglobin were added to the reaction mixture.

The analysis under the conditions given revealed a p50 value of 21.6 mm of Hg and an n50 value of 1.6.

### EXAMPLE 10

### Preparation of a Cross-Linked Pig Hemoglobin with the Addition of an Inventive, Unreactive Effector

The product was prepared as in Example 7 with the difference that 2 moles of mellitic acid per mole of hemoglobin were added to the reaction mixture.

The analysis under the conditions given revealed a p50 value of 16.5 mm of Hg and an n50 value of 1.5.

### EXAMPLE 11

### Comparison

### Preparation of a Cross-Linked Human Hemoglobin Without the Addition of an Unreactive Effector

The product was prepared as in Example 3 with the difference that human hemoglobin was used.

The analysis under the conditions given revealed a p50 value of 20 mm of Hg and an n50 value of 1.5.

### EXAMPLE 12

### Preparation of a Cross-Linked Human Hemoglobin with the Addition of an Inventive, Unreactive Effector

The product was prepared as in Example 11, with the difference that 2 moles of bisphosphoglycerate per mole of hemoglobin were added to the reaction mixture.

The analysis under the conditions given revealed a p50 value of 13 mm of Hg and an n50 value of 1.4.

### EXAMPLE 13

### Preparation of a Cross-Linked Human Hemoglobin with the Addition of an Inventive, Unreactive Effector

The product was prepared as in Example 11, with the difference that 2 moles of inositol hexaphosphate per mole of hemoglobin were added to the reaction mixture.

The analysis under the conditions given revealed a p50 value of 12 mm of Hg and an n50 value of 1.8.

1. A method for the preparation of artificial oxygen carriers from chemically cross-linked hemoglobin with improved functional properties, wherein, before the covalent cross-linking of the hemoglobin, chemically unreactive effectors of the oxygen affinity of the hemoglobin are the added to the reaction solution of the latter.

2. The method of claim 1, wherein 2,3-bisphosphoglycerate, inositol hexaphosphate, inositol hexasulfate or mellitic acid are used as effectors of the oxygen affinity of the hemoglobin.

3. The method of claim 2, wherein 2,3-bisphosphoglycerate is used as unreactive effector.

4. The method of one of the claims 1 to 3, wherein the unreactive effector of the oxygen affinity of the hemoglobin is added in an amount of 1 to 20 moles per mole of hemoglobin before the latter is cross-linked.

5. The method of one of the claims 1 to 4, wherein the hemoglobin originates from man, from pigs or from cattle.

6. The method of one of the claims 1 to 5, wherein the hemoglobin is used in a concentration of 10 to 420 g/L,

7. The method of one of the claims 1 to 6, wherein, for crosslinking in the presence of the chemically unreactive effector of the oxygen affinity, native hemoglobin or a hemoglobin, derivatized by a prior chemical modification, is used.

8. The method of one of the claims 1 to 7, wherein the hemoglobin is used in the deoxygenated state.

9. The method of one of the claims 1 to 8, wherein the hemoglobin is cross-linked by means of a difunctional or polyfunctional cross-linking agent for proteins.

10. The method of one of the claims 1 to 9, wherein the crosslinking agent is used in an amount of 3 to 60 moles per mole of monomeric hemoglobin.

11. The method of one of the claims 1 to 10, wherein the hemoglobin is cross-linked by means of a difunctional cross-linking agent for proteins, selected from butadiene diepoxide, divinylsulfone, a diisocyanate, especially hexamethylene diisocyanate, cyclohexyl diisocyanate and 2,5-bisisocyanatobenzenesulfonic acid, a di-N-hydroxysuccinimidyl ester, a diimido ester, or a dialdehyde, especially glyoxal, the similarly reacting glycol aldehyde or glutardialdehyde.

12. The method of claim 11, wherein the difunctional cross-linking agent is glutardialdehyde.

13. The method of one of the claims 1 to 12, wherein the crosslinked hemoglobin, treated during the cross-linking with a chemically unreactive effector, optionally is modified further chemically and derivatized.

14. The method of claim 13, wherein the cross-linked hemoglobin is linked covalently to a polyalkylene oxide.

15. The method of claim 14, wherein the cross-linked hemoglobin is linked covalently to a polyethylene oxide.

16. The method of one of the claims 1 to 15, wherein the crosslinked hemoglobin which optionally has been modified further chemically and has been treated with an unreactive effector, is purified preparatively by dialysis, centrifugation, clarifying filtration and/or preparative chromatographic graphic methods.

17. The use of a cross-linked hemoglobin, prepared by the methods of one of the claims 1 to 16, for preparing an agent for intravasal or biomedical use as artificial oxygen carrier.

18. The use of claim 17, wherein the agent is used in the form of a pharmaceutical preparation as a replacement for blood or as an addition to the blood or to a nutrient solution, in the human and animal organism, in individual organs, or in biotechnical applications.
